(19) United States
(12) Patent Application Publication
(10) Pub. No.: US 2026/0200851 A1
(43) Pub. Date: Jul. 16, 2026

(54) ORGANIC IONIC PLASTIC CRYSTALS AS ELECTROLYTES

(71) Applicant: THE BOARD OF REGENTS OF THE NEVADA SYSTEM OF HIGHER EDUCATION ON BEHALF OF THE UNIVERSITY OF, Las Vegas, NV (US)

(72) Inventors: Pradip K. Bhowmik, Las Vegas, NV (US); Haesook Han, Las Vegas, NV (US)

(21) Appl. No.: 19/133,690

(22) PCT Filed: Nov. 28, 2023
(86) PCT No.: PCT/US2023/081258

§ 371 Date: May 28, 2025

Related U.S. Application Data
(60) Provisional application No. 63/428,564, filed on Nov. 29, 2022.

(51) Int. Cl. C07D 213/04; H01G 9/20; H01G 11/56; H01M 10/056

(52) U.S. Cl. CPC C07D 213/04; H01G 9/2009; H01G 11/56; H01M 10/056; H01M 2300/0065

(57) ABSTRACT

Described herein are organic ion plastic crystals (OIPCs) comprising extended viologen salts. In one aspect, OIPCs can C1 be used in solid state batteries, supercapacitors, solar cells, or perovskite photovoltaic cells.

### CROSS-REFERENCE TO RELATED APPLICATIONS

This application claims priority to U.S. Provisional Application No. 63/428,564, filed on Nov. 29, 2022, which is incorporated by reference herein in its entirety.

### TECHNICAL FIELD

Described herein are organic ion plastic crystals (OIPCs) comprising dicationic extended viologens with bistriflimide, bistriflate or bisnonaflate anions. In one aspect, OPCs can be used in solid state batteries, supercapacitors, solar cells, or perovskite photovoltaic cells.

### BACKGROUND

Lithium-ion batteries (LIBs) are the crucial components as energy-storage devices in many consumer electronics including, laptops and smartphones. Demands for their large-scale utilizations are ever-increasing growth along with a rapid expansion of the electric vehicles (EVs) market. Current LIBs are the fruits of manufacturers' labor and are practically safe as final products, which ensure sufficient precautions at each scale from optimization of cell chemistry to mechanical crash protection of battery modules. However, at the cell-chemistry level, LIBs rely on flammable organic liquid electrolytes, posing inherent safety risks.

Therefore, solid-state batteries (SSBs), in which solid electrolytes are used as ion-conductive pathways have attracted attention because of the higher thermal stability of solid electrolytes compared to conventional liquid electrolytes in LIBs. If SSBs can be made cost-effective and achieve comparable performance with LIBs, the replacement of LIBs with SSBs will be viable to develop this new technology. Another important factor is to consider for the process applicability of solid-state electrodes comprising active materials and solid electrolytes to current LIB production lines. Highly compatible preparation methods for the electrodes are beneficial to boost a wide adaptation of SSBs in energy storage applications.

Although inorganic solid electrolytes are promising materials in terms of their bulk ionic conductivities, they require additional steps to form void-free contacts between the solid electrolytes and active materials. They are processed at relatively high temperatures (1000° C.) and pressures (350 MPa). Such high temperature and pressure procedures cannot be directly performed in the roll-to-roll electrode fabrication process and, thus, increase costs for the mass production of SSBs. In this context, organic ionic solid electrolytes also known as organic ion plastic crystals (OIPCs) become alternative, attractive candidates because of their excellent process applicability. If intelligently designed OIPCs, like current LIB fabrication technologies, can be simply employed without implementing any additional low-throughput processes.

However, a significant challenge associated with OIPCs is achieving high ionic conductivity in the solid state. OIPCs are one class of solid-state electrolyte, composed entirely of ions with the advantageous properties observed in ionic liquids (ILs) such as nonflammability, high conductivity, and the ability to form stable and highly conductive solid electrolyte interface (SEI) layers in lithium cells. OIPCs exist as disordered solids at room temperature and tend to have smaller cations compared to ILs. OIPCs are usually synthesized from pyrrolidinium, ammonium, phosphonium, and imidazolium all of which are monocationic and fluorinated anions that include bistriflimide (NTf₂)⁻, trifluoromethane sulfonate (OTf)⁻, perfluorobutanesulfonate (ONf)⁻, BF₄⁻, and PF₆⁻, among others. OIPCs are solid-state analogues of ionic liquids (ILs) that inherit advantages from their ionic nature including low flammability, negligible vapor pressure, and high thermal and chemical stability. Smaller cations allow for more efficient packing of the ions to increase the melting point and long-range order while generally maintaining the short-range disorder required for plasticity.

Plastic behavior stems from OIPCs undergoing one or more solid-solid phase transitions before melting. In a disordered plastic material, the solid phase before melting is entropically close to the liquid phase, as evidenced by a small entropy of fusion. This means that OIPCs tend to be soft and malleable, which allows better contact with electrodes than other, more brittle solid-state inorganic electrolytes such as ceramics and is conducive for better ionic conductivity. The library of cations (vide supra) known to form OIPCs however, is far smaller than for ILs, and the development of new cations, specially dicationic OIPCs, is critical to advance their use as solid electrolytes. The measured ionic conductivity of dicationic OIPCs in the solid state is higher than monocationic OIPCs.

What is needed are organic ion plastic crystals (OPCs) for use in solid state batteries, supercapacitors, solar cells, or perovskite photovoltaic cells.

### SUMMARY

One embodiment described herein is a solid-state electrolyte comprising:
a viologen salt of formula (I):

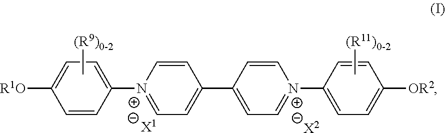

wherein:
^{Θ}X¹ and ^{Θ}X² are each independently

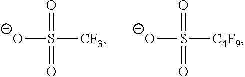

^{Θ}BF₄, ^{Θ}PF₆, ^{Θ}Br, ^{Θ}Cl, or

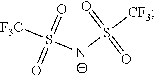 [SMILES: O=S(=O)([N-]S(=O)(=O)C(F)(F)F)C(F)(F)F]

R¹ and R² are each independently C₄₋₂₀alkyl or

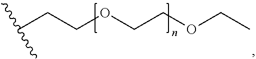

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.

In one aspect, R¹ is

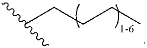

In another aspect, R² is

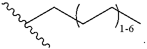

In another aspect, the viologen salt is a symmetric viologen salt.

In another aspect, ^{Θ}X¹ and ^{Θ}X² are each

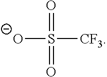

In another aspect, ^{Θ}X¹ and ^{Θ}X² are each

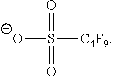

In another aspect, the electrolyte comprises a viologen salt of formula (I-a):

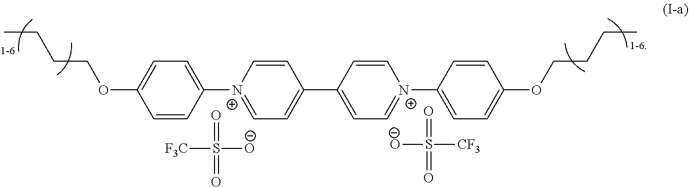

In another aspect, the electrolyte comprises a viologen salt of formula (I-b):

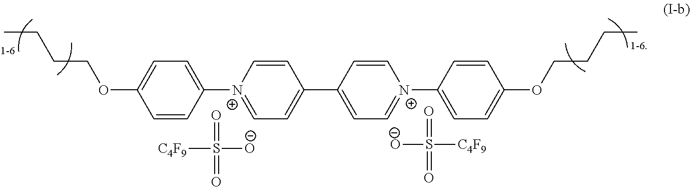

In another aspect, a solid-state battery, supercapacitor, solar cell, or perovskite photovoltaic cell comprises the solid-state electrolyte comprising a viologen salt of formula (I).

Another embodiment described herein is a solid-state battery comprising:
a cathode;
an anode;
a separator; and
a solid-state electrolyte, wherein the solid-state electrolyte comprises:
a viologen salt of formula (I):

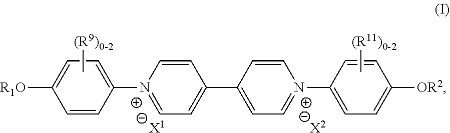

wherein:
^{Θ}X¹ and ^{Θ}X² are each independently

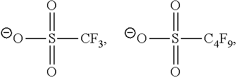

^{Θ}BF₄, ^{Θ}PF₆, ^{Θ}Br, ^{Θ}Cl, or

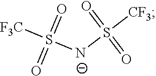 [SMILES: O=S(=O)([N-]S(=O)(=O)C(F)(F)F)C(F)(F)F]

R¹ and R² are each independently C₄₋₂₀alkyl or

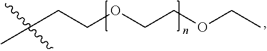

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.

Another embodiment described herein is a supercapacitor comprising:
a cathode;
an anode;
a separator; and
a solid-state electrolyte, wherein the solid-state electrolyte comprises:
a viologen salt of formula (I):

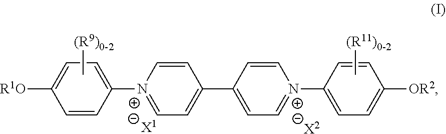

wherein:
^{Θ}X¹ and ^{Θ}X² are each independently

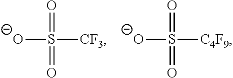

^{Θ}BF₄, ^{Θ}PF₆, ^{Θ}Br, ^{Θ}Cl, or

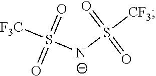 [SMILES: O=S(=O)([N-]S(=O)(=O)C(F)(F)F)C(F)(F)F]

R¹ and R² are each independently C₄₋₂₀alkyl or

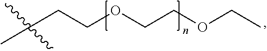

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.

Another embodiment described herein is a solar cell comprising:
a n-type semiconductor layer;
a p-type semiconductor layer; and
a solid-state electrolyte, wherein the solid-state electrolyte comprises:
a viologen salt of formula (I):

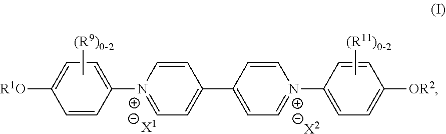

wherein:
^{Θ}X¹ and ^{Θ}X² are each independently

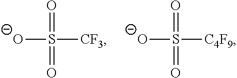

^{Θ}BF₄, ^{Θ}PF₆, ^{Θ}Br, ^{Θ}Cl, or

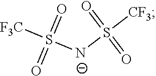 [SMILES: O=S(=O)([N-]S(=O)(=O)C(F)(F)F)C(F)(F)F]

R¹ and R² are each independently C₄₋₂₀alkyl or

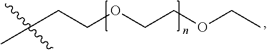

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.

Another embodiment described herein is a perovskite photovoltaic cell comprising:
a n-type semiconductor layer;
an electron transport layer (ETL);
a p-type semiconductor layer; and
a solid-state electrolyte, wherein the solid-state electrolyte comprises:
a viologen salt of formula (I):

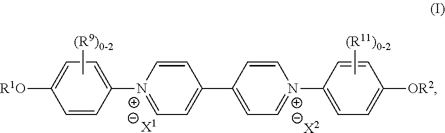

wherein:
^{Θ}X¹ and ^{Θ}X² are each independently

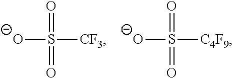

^{Θ}BF₄, ^{Θ}PF₆, ^{Θ}Br, ^{Θ}Cl, or

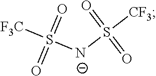 [SMILES: O=S(=O)([N-]S(=O)(=O)C(F)(F)F)C(F)(F)F]

R¹ and R² are each independently C₄₋₂₀alkyl or

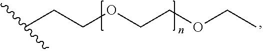

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.

In another aspect, the solid-state battery, supercapacitor, solar cell, or perovskite photovoltaic cell comprises a viologen salt of formula (I) wherein R¹ is

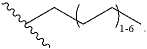

In another aspect, the solid-state battery, supercapacitor, solar cell, or perovskite photovoltaic cell comprises a viologen salt of formula (I) wherein R² is

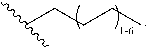

In another aspect, the solid-state battery, supercapacitor, solar cell, or perovskite photovoltaic cell comprises a viologen salt of formula (I) wherein the viologen salt is a symmetric viologen salt.

In another aspect, the solid-state battery, supercapacitor, solar cell, or perovskite photovoltaic cell comprises a viologen salt of formula (I) wherein ^{Θ}X¹ and ^{Θ}X² are each

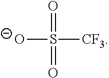

In another aspect, the solid-state battery, supercapacitor, solar cell, or perovskite photovoltaic cell comprises a viologen salt of formula (I) wherein ^{Θ}X¹ and ^{Θ}X² are each

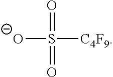

In another aspect, the electrolyte of the solid-state battery, supercapacitor, solar cell, or perovskite photovoltaic cell comprises a viologen salt of formula (I-a):

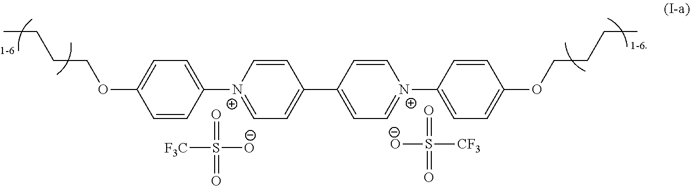

In another aspect, the electrolyte of the solid-state battery, supercapacitor, solar cell, or perovskite photovoltaic cell comprises a viologen salt of formula (I-b):

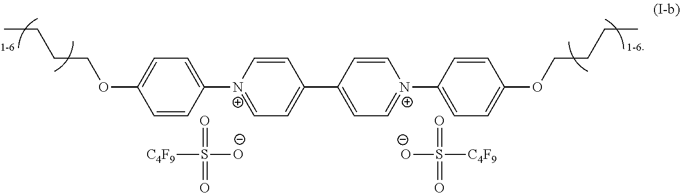

### DESCRIPTION OF THE DRAWINGS

FIG. 1A-C show ¹H NMR (FIG. 1A), ¹³C NMR (FIG. 1B) and ¹⁹F NMR (FIG. 1C) spectra of EVₙSO₃CF₃, where EVₙ=C₁₂H₂₅ (EV12), in CD₃OD taken at room temperature.

FIG. 2A-C show ¹H NMR (FIG. 2A), ¹³C NMR (FIG. 2B), and ¹⁹F NMR (FIG. 2C) spectra of EVₙSO₃C₄F₉, where EVₙ=C₁₂H₂₅ (EV12), in CD₃OD taken at room temperature.

FIG. 3 shows differential scanning calorimetry (DSC) thermograms of EVₙSO₃CF₃, where EVₙ=C₁₀H₂₁ (EV10), obtained at heating and cooling rates of 10° C./min.

FIG. 4 shows DSC thermograms of EVₙSO₃CF₃, where EVₙ=C₁₂H₂₅ (EV12), obtained at heating and cooling rates of 10° C./min.

FIG. 5 shows DSC thermograms of EVₙSO₃C₄F₉, where EVₙ=C₁₀H₂₁ (EV10), obtained at heating and cooling rates of 10° C./min.

FIG. 6 shows DSC thermograms of EVₙSO₃C₄F₉, where EVₙ=C₁₂H₂₅ (EV12), obtained at heating and cooling rates of 10° C./min.

FIG. 7 shows thermogravimetric analysis (TGA) thermograms of EVₙSO₃CF₃, where EVₙ=C₆H₁₃ (EV6), C₇H₁₅ (EV7), C₁₂H₂₅ (EV12), and C₁₄H₂₉ (EV14), obtained at a heating rate of 10° C./min in nitrogen.

FIG. 8 shows TGA thermograms of EVₙSO₃C₄F₉, where EVₙ=C₆H₁₃ (EV6), C₈H₁₇ (EV8), C₁₀H₂₁ (EV10), and C₁₂H₂₅ (EV12), obtained at a heating rate of 10° C./min in nitrogen.

### DETAILED DESCRIPTION

Unless otherwise defined, all technical and scientific terms used herein have the same meaning as commonly understood by one of ordinary skill in the art. For example, any nomenclatures used in connection with, and techniques of synthetic chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein.

Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched chain hydrocarbon of 1 to 10 carbon atoms, for example, of 2 to 5 carbon atoms. Representative examples of alkylene include, but are not limited to, —CH₂—, —CD₂-, —CH₂CH₂—, —CH₂CH₂CH₂—, —CH₂CH₂CH₂CH₂—, and —CH₂CH₂CH₂CH₂CH₂—.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl).

Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl).

Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

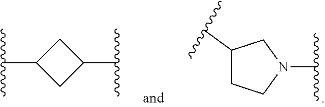 [SMILES: *C1CC(*)C1 ; *C1CCN(*)C1]

Cycloalkylene and heterocyclylene include a geminal divalent groups such as 1,1-C₃₋₈cycloalkylene

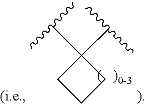

A further example is 1,1-cyclopropylene

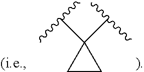 [SMILES: *C1(*)CC1]

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “fluoroalkylene,” as used herein, means an alkylene group, as defined herein, in which one, two, three, four, five, six, seven or eight hydrogen atoms are replaced by fluorine. Representative examples of fluoroalkyl include, but are not limited to —CF₂—, —CH₂CF₂—, 1,2-difluoroethylene, 1,1,2,2-tetrafluoroethylene, 1,3,3,3-tetrafluoropropylene, 1,1,2,3,3-pentafluoropropylene, and perfluoropropylene such as 1,1,2,2,3,3-hexafluoropropylene.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl).

A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

The term “n-type semiconductor,” as used herein, means a semiconductor where the electrons are majority charge carriers and holes are minority charge carriers.

The term “p-type semiconductor,” as used herein, means a semiconductor where the holes are majority charge carriers and electrons are minority charge carriers.

Described herein are a series of organic ion plastic crystals (OIPCs) having high ionic conductivity in the solid state for next generation of energy storage devices that are capable of higher energy density and superior safety performance when compared with the state-of-the art LIBs. They are based on extended viologens consisting of dicationic charges with bistriflate or bisnonaflate ions the typical structures of which are given in Schemes 1 and 2.

OIPCs are an important class of solid-state electrolytes with significant advantages over flammable organic electrolytes and ionic liquid electrolytes including plasticity, nonflammability and high ionic conductivity. OIPCs can be used in emerging applications in electrochemical devices, including solid state batteries, lithium ion, sodium ion batteries, fuel cells and dye-sensitized solar cells. OIPCs can also be employed as additives in perovskite solar cells enhancing power conversion efficiencies and stability. OIPCs are useful for the preparation of flexible and quasi-solid state gel polymer electrolytes for application in carbon supercapacitors and OIPCs prevent the dendritic growth associated with LIBs.

One embodiment described herein is a solid-state electrolyte comprising:
a viologen salt of formula (I):

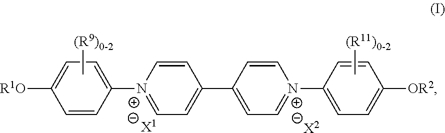

wherein:
^{Θ}X¹ and ^{Θ}X² are each independently

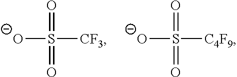

^{Θ}BF₄, ^{Θ}PF₆, ^{Θ}Br, ^{Θ}Cl, or

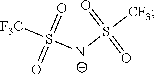 [SMILES: O=S(=O)([N-]S(=O)(=O)C(F)(F)F)C(F)(F)F]

R¹ and R² are each independently C₄₋₂₀alkyl or

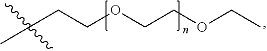

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.

In one aspect, R¹ is

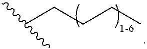

In another aspect, R² is

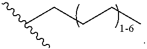

In another aspect, the viologen salt is a symmetric viologen salt.

In another aspect, ^{Θ}X¹ and ^{Θ}X² are each

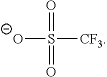

In another aspect, ^{Θ}X¹ and ^{Θ}X² are each

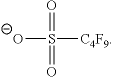

In another aspect, the electrolyte comprises a viologen salt of formula (I-a):

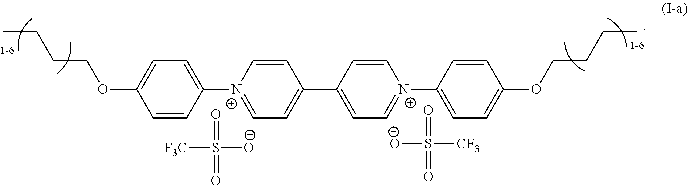

In another aspect, the electrolyte comprises a viologen salt of formula (I-b):

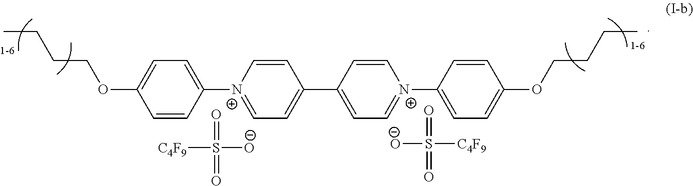

In another aspect, a solid-state battery, supercapacitor, solar cell, or perovskite photovoltaic cell comprises the solid-state electrolyte comprising a viologen salt of formula (I).

Another embodiment described herein is a solid-state battery comprising:
a cathode;
an anode;
a separator; and
a solid-state electrolyte, wherein the solid-state electrolyte comprises:
a viologen salt of formula (I):

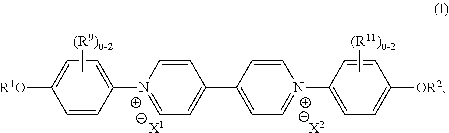

wherein:
^{Θ}X¹ and ^{Θ}X² are each independently

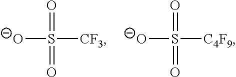

^{Θ}BF₄, ^{Θ}PF₆, ^{Θ}Br, ^{Θ}Cl, or

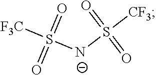 [SMILES: O=S(=O)([N-]S(=O)(=O)C(F)(F)F)C(F)(F)F]

R¹ and R² are each independently C₄₋₂₀alkyl or

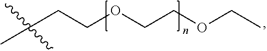

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.

Another embodiment described herein is a supercapacitor comprising:
a cathode;
an anode;
a separator; and
a solid-state electrolyte, wherein the solid-state electrolyte comprises:
a viologen salt of formula (I):

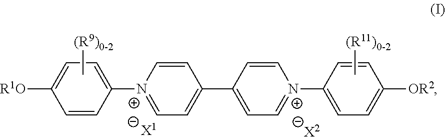

wherein:

^{Θ}X¹ and ^{Θ}X² are each independently

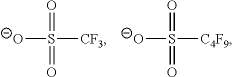

^{Θ}BF₄, ^{Θ}PF₆, ^{Θ}Br, ^{Θ}Cl, or

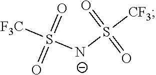 [SMILES: O=S(=O)([N-]S(=O)(=O)C(F)(F)F)C(F)(F)F]

R¹ and R² are each independently C₄₋₂₀alkyl or

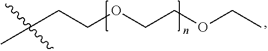

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.

Another embodiment described herein is a solar cell comprising:
a n-type semiconductor layer;
a p-type semiconductor layer; and
a solid-state electrolyte, wherein the solid-state electrolyte comprises:
a viologen salt of formula (I):

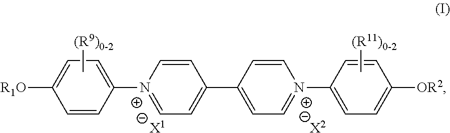

wherein:
^{Θ}X¹ and ^{Θ}X² are each independently

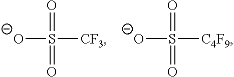

^{Θ}BF₄, ^{Θ}PF₆, ^{Θ}Br, ^{Θ}Cl, or

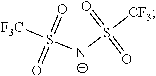 [SMILES: O=S(=O)([N-]S(=O)(=O)C(F)(F)F)C(F)(F)F]

R¹ and R² are each independently C₄₋₂₀alkyl or

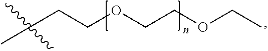

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.

Another embodiment described herein is a perovskite photovoltaic cell comprising:
a n-type semiconductor layer;
an electron transport layer (ETL);
a p-type semiconductor layer; and
a solid-state electrolyte, wherein the solid-state electrolyte comprises:
a viologen salt of formula (I):

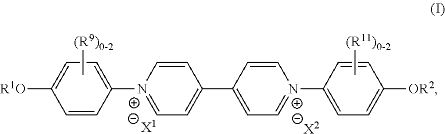

wherein:
^{Θ}X¹ and ^{Θ}X² are each independently

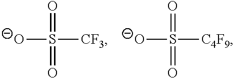

^{Θ}BF₄, ^{Θ}PF₆, ^{Θ}Br, ^{Θ}Cl, or

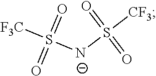 [SMILES: O=S(=O)([N-]S(=O)(=O)C(F)(F)F)C(F)(F)F]

R¹ and R² are each independently C₄₋₂₀alkyl or

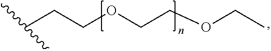

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.

In another aspect, the solid-state battery, supercapacitor, solar cell, or perovskite photovoltaic cell comprises a viologen salt of formula (I) wherein R¹ is

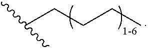

In another aspect, the solid-state battery, supercapacitor, solar cell, or perovskite photovoltaic cell comprises a viologen salt of formula (I) wherein R² is

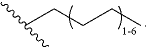

In another aspect, the solid-state battery, supercapacitor, solar cell, or perovskite photovoltaic cell comprises a viologen salt of formula (I) wherein the viologen salt is a symmetric viologen salt.

In another aspect, the solid-state battery, supercapacitor, solar cell, or perovskite photovoltaic cell comprises a viologen salt of formula (I) wherein ^{Θ}X¹ and ^{Θ}X² are each

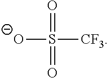

In another aspect, the solid-state battery, supercapacitor, solar cell, or perovskite photovoltaic cell comprises a viologen salt of formula (I) wherein ^{Θ}X¹ and ^{Θ}X² are each

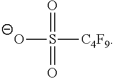

In another aspect, the electrolyte of the solid-state battery, supercapacitor, solar cell, or perovskite photovoltaic cell comprises a viologen salt of formula (I-a):

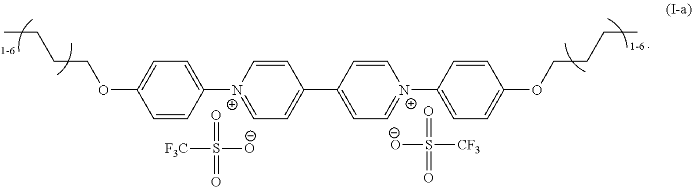

In another aspect, the electrolyte of the solid-state battery, supercapacitor, solar cell, or perovskite photovoltaic cell comprises a viologen salt of formula (I-b):

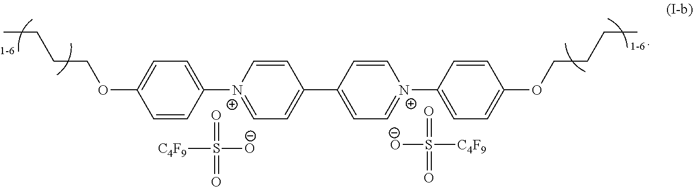

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A solid-state electrolyte comprising:
   a viologen salt of formula (I):

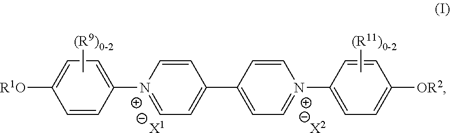

wherein:
^{Θ}X¹ and ^{Θ}X² are each independently

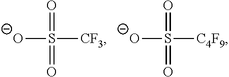

^{Θ}BF₄, ^{Θ}PF₆, ^{Θ}Br, ^{Θ}Cl, or

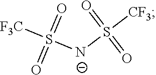 [SMILES: O=S(=O)([N-]S(=O)(=O)C(F)(F)F)C(F)(F)F]

R¹ and R² are each independently C₄₋₂₀alkyl or

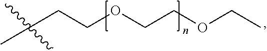

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.
Clause 2. The solid-state electrolyte of clause 1, wherein R¹ is

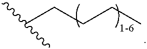

Clause 3. The solid-state electrolyte of clause 1 or 2, wherein R² is

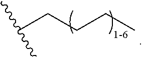

Clause 4. The solid-state electrolyte of any one of clauses 1-3, wherein the viologen salt is a symmetric viologen salt.
Clause 5. The solid-state electrolyte of clause 1, wherein ^{Θ}X¹ and ^{Θ}X² are each

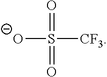

Clause 6. The solid-state electrolyte of clause 1, wherein ^{Θ}X¹ and ^{Θ}X² are each

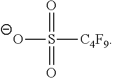

Clause 7. The solid-state electrolyte of any one of clauses 1-5, comprising a viologen salt of formula (I-a):

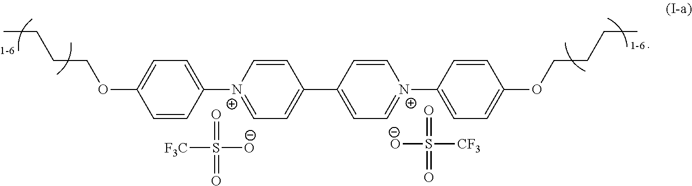

Clause 8. The solid-state electrolyte of any one of clauses 1-4 or 6, comprising a viologen salt of formula (I-b):

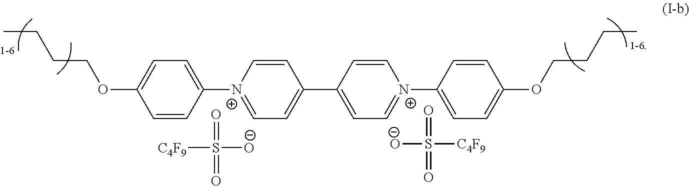

Clause 9. A solid-state battery, supercapacitor, solar cell, or perovskite photovoltaic cell comprising the solid-state electrolyte of any one of clauses 1-8.
Clause 10. A solid-state battery comprising
   a cathode;
   an anode;
   a separator; and
   a solid-state electrolyte, wherein the solid-state electrolyte comprises:
   a viologen salt of formula (I):

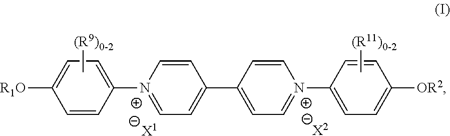

wherein:
^{Θ}X¹ and ^{Θ}X² are each independently

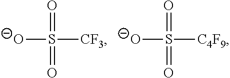

^{Θ}BF₄, ^{Θ}PF₆, ^{Θ}Br, ^{Θ}Cl, or

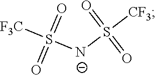 [SMILES: O=S(=O)([N-]S(=O)(=O)C(F)(F)F)C(F)(F)F]

R¹ and R² are each independently C₄₋₂₀alkyl or

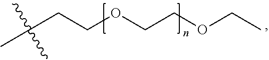

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.
Clause 11. A supercapacitor comprising:
   a cathode;
   an anode;
   a separator; and
   a solid-state electrolyte, wherein the solid-state electrolyte comprises:
   a viologen salt of formula (I):

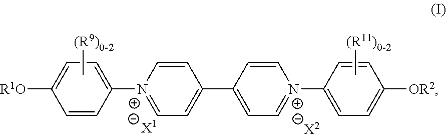

wherein:
^{Θ}X¹ and ^{Θ}X² are each independently

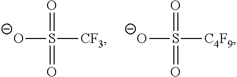

^{Θ}BF₄, ^{Θ}PF₆, ^{Θ}Br, ^{Θ}Cl, or

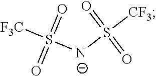 [SMILES: O=S(=O)([N-]S(=O)(=O)C(F)(F)F)C(F)(F)F]

R¹ and R² are each independently C₄₋₂₀alkyl or

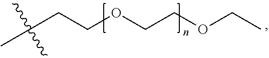

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.
Clause 12. A solar cell comprising:
   a n-type semiconductor layer,
   a p-type semiconductor layer; and
   a solid-state electrolyte, wherein the solid-state electrolyte comprises:
   a viologen salt of formula (I):

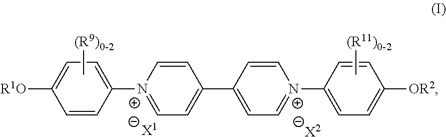

wherein:
^{Θ}X¹ and ^{Θ}X² are each independently

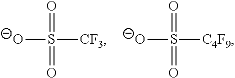

^{Θ}BF₄, ^{Θ}PF₆, ^{Θ}Br, ^{Θ}Cl, or

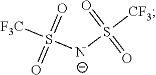 [SMILES: O=S(=O)([N-]S(=O)(=O)C(F)(F)F)C(F)(F)F]

R¹ and R² are each independently C₄₋₂₀alkyl or

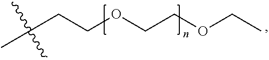

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.
Clause 13. A perovskite photovoltaic cell comprising:
   a n-type semiconductor layer;
   an electron transport layer (ETL);
   a p-type semiconductor layer; and
   a solid-state electrolyte, wherein the solid-state electrolyte comprises:
   a viologen salt of formula (I):

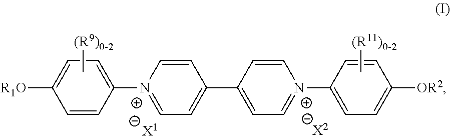

wherein:
^{Θ}X¹ and ^{Θ}X² are each independently

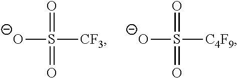

^{Θ}BF₄, ^{Θ}PF₆, ^{Θ}Br, ^{Θ}Cl, or

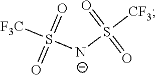 [SMILES: O=S(=O)([N-]S(=O)(=O)C(F)(F)F)C(F)(F)F]

R¹ and R² are each independently C₄₋₂₀alkyl or

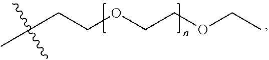

where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.
Clause 14. The solid-state battery, supercapacitor, solar cell, or perovskite photovoltaic cell of any one of clauses 10-13, wherein R¹ is

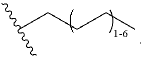

Clause 15. The solid-state battery, supercapacitor, solar cell, or perovskite photovoltaic cell of any one of clauses 10-14, wherein R² is

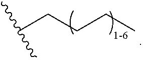

Clause 16. The solid-state battery, supercapacitor, solar cell, or perovskite photovoltaic cell of any one of clauses 10-15, wherein the viologen salt is a symmetric viologen salt.
Clause 17. The solid-state battery, supercapacitor, solar cell, or perovskite photovoltaic cell of any one of clauses 10-16, wherein ^{Θ}X¹ and ^{Θ}X² are each

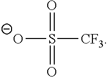

Clause 18. The solid-state battery, supercapacitor, solar cell, or perovskite photovoltaic cell of any one of clauses 10-16, wherein ^{Θ}X¹ and ^{Θ}X² are each

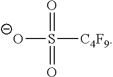

Clause 19. The solid-state battery, supercapacitor, solar cell, or perovskite photovoltaic cell of any one of clauses 10-17, wherein the electrolyte comprises a viologen salt of formula (I-a):

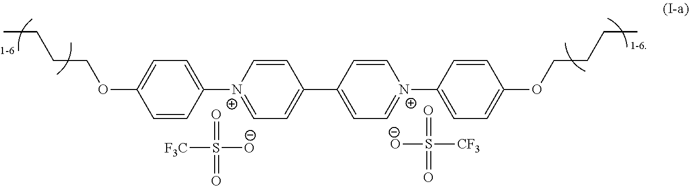

Clause 20. The solid-state battery, supercapacitor, solar cell, or perovskite photovoltaic cell of any one of clauses 10-16 or 18, wherein the electrolyte comprises a viologen salt of formula (I-b):

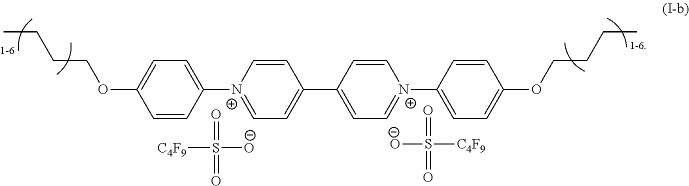

### EXAMPLES

### Materials and Methods

All chemicals and solvents were reagent grade and purchased from commercial vendors (Acros Organics, Alfa-Aesar, Sigma-Aldrich, and TCI America) and were used as received.

The salt samples were prepared in thin films casting from chloroform on NaCl plates and subsequently vacuum dried at 70° C. overnight. The ¹H, ¹³C, and ¹⁹F nuclear magnetic resonance (NMR) sample solutions of extended viologen salts of bistriflate and bisnonaflate were prepared by dissolving 10 mg of each of the salts in 1 mL CD₃OD, and the spectra were recorded by using a VNMR 400 spectrometer operating at 400, 100, 376 MHz, respectively, at room temperature and chemical shifts were referenced to tetramethylsilane (TMS) for ¹H and ¹³C nuclei and trichlorofluoromethane (CFCl₃) for ¹⁹F nuclei, respectively. Elemental analyses were performed by Atlantic Microlab Inc., Norcross, GA.

The phase transition temperatures of the salts were conducted on TA module differential scanning calorimetry DSC Q200 series in nitrogen at heating and cooling rates of 10° C.·min⁻¹. The temperature axis of the DSC thermograms was calibrated with reference standards of high purity indium and tin. The thermal stability properties of these salts were conducted using a thermogravimetric analysis (TGA) Q50 instrument at a heating rate of 10° C.·min⁻¹ in nitrogen.

### Example 1

### Synthetic Procedure for 4-Oligoethyleneoxy Anilines

4-(2-Ethoxyethoxy) aniline, 4-(2-(2-ethoxyethoxy) ethoxy] aniline, and 4-(2-(2-(2-ethoxyethoxy) ethoxy)ethoxy]ethoxy]aniline were prepared according to the procedure described hereafter. Acetone and oligoethylene bromides were used in the alkylation of 4-hydroxyacetanilide. Oligoethylene bromides were prepared via Appel reaction (Scheme 1, Step 1). As all the three aforementioned anilines were prepared in an identical manner, only the synthesis of 4-[2-[2-(2-ethoxyethoxy) ethoxy)ethoxy]ethoxy]aniline is described below in detail. This aniline was prepared in a three-step reaction, starting with the bromination of 4-[2-(2-ethoxyethoxy) ethoxy]ethanol via Appel reaction.

Scheme 1 shows the chemical structures of extended 4-oligoethyleneoxy aniline viologen salts (OIPCs) prepared by using Zincke Salt and metathesis reactions.

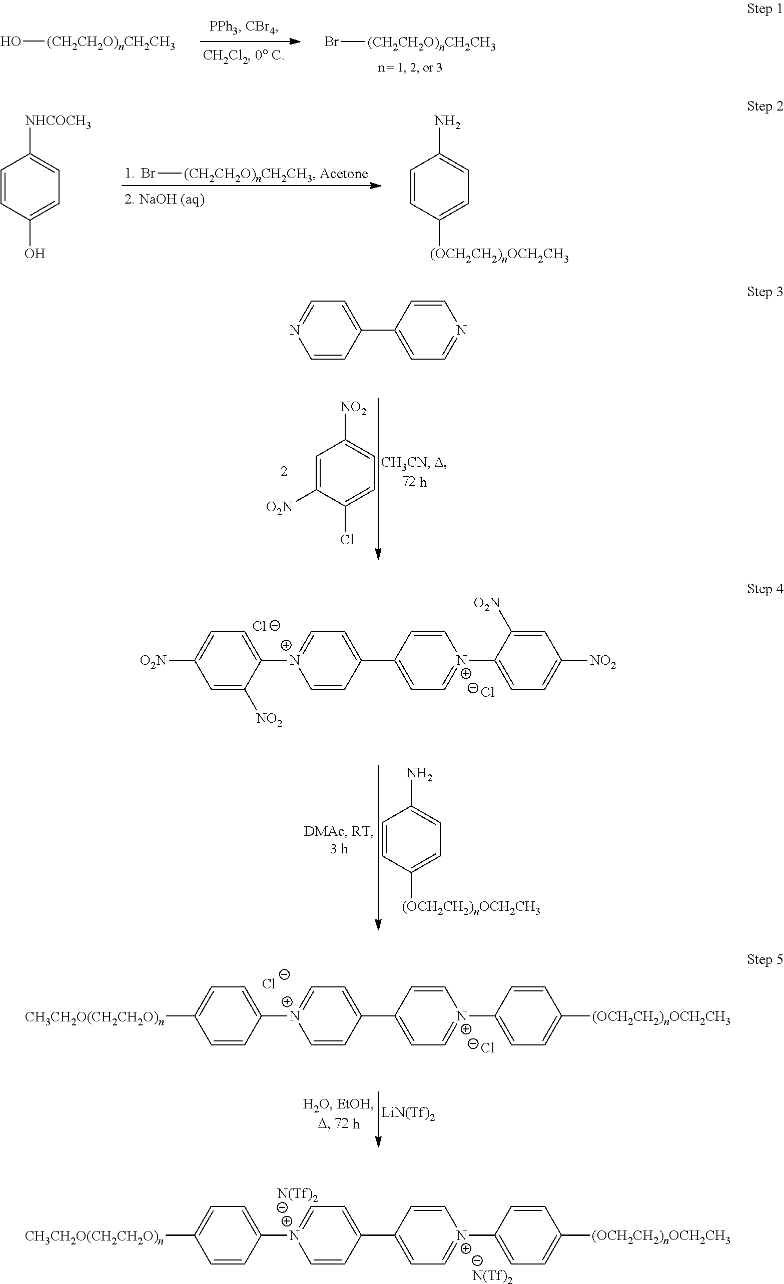

For the bromination of 4-[2-(2-ethoxyethoxy) ethoxy]ethanol via Appel reaction, the stoichiometric amount of 4-[2-(2-ethoxyethoxy) ethoxy]ethanol (1.000 g, 5.61 mmol) and excess amount of triphenylphosphine (2.458 g, 9.37 mmol) were added to an Erlenmeyer flask and dissolved in 10 mL of dichloromethane (DCM). When the mixture was stirred and cooled to 0° C., carbon tetrabromide (2.326 g, 7.01 mmol) dissolved in 10 mL of DCM was added to this mixture dropwise. Upon the complete addition of carbon tetrabromide solution, the reaction mixture was stirred at room temperature for 30 min. Subsequently, the DCM was removed using a rotary evaporator. After the removal of DCM, 30 mL of hexane was added to the reaction mixture to precipitate out the excess starting material and byproducts. The reaction mixture (containing hexane) was cooled down to −77° C. by keeping the flask in an isopropyl alcohol and dry ice bath. The contents of the flask were filtered through Celite, and the hexane was evaporated, leaving behind the pure product of 4-[2-(2-ethoxyethoxy) ethoxy]ethyl bromide (0.700 g, 2.90 mmol, Yield: 52%).

In the Scheme 1, Step 2, the alkylation of 4-hydroxyacetanilide was performed as follows. 4-[2-(2-ethoxyethoxy) ethoxy]bromide (0.700 g, 2.90 mmol) was added to a round-bottomed flask containing 4-hydroxyacetanilide (0.483 g, 3.19 mmol) dissolved in 50 mL of acetone. Potassium carbonate (0.401 mg, 2.90 mmol) was added to the flask and the reaction mixture was heated to reflux on stirring for 24 h. At the end of the reaction, the mixture was brought to room temperature and filtered. The acetone was removed using a rotary evaporator and the product was purified by extraction with DCM and warm deionized water. The DCM was then evaporated to yield a pure product of 4-[2-[2-(2-ethoxyethoxy) ethoxy]ethoxy]acetanilide (0.800 g, 2.57 mmol, Yield: 88%).

Finally, a hydrolysis reaction (Scheme 1, Step 2) was performed by adding 4-[2-[2-(2-ethoxyethoxy) ethoxy]ethoxy]acetanilide (0.800 g, 2.57 mmol) to a three-necked flask with sodium hydroxide (2.000 g, 50.0 mmol) dissolved in 25 mL of deionized water. The reaction flask was heated under nitrogen atmosphere for 12 h. At the end of the reaction, the flask was cooled down to room temperature and the desired product was purified by extraction with DCM and deionized water. The DCM was removed using a rotary evaporator to yield a pure product of 4-[2-[2-(2-ethoxyethoxy) ethoxy]ethoxy]aniline (0.576 g, 2.14 mmol, Yield: 83%).

### Synthetic Procedure for Zincke Salts

The Zinckes salt were prepared, by the reaction of 1-chloro-2,4-dinitrobenzene (2.5 equivalents) with 4,4′-bipyridine (1 equiv.) on heating to reflux at 81° C. in acetonitrile (Scheme 1, Step 3).

### Synthetic Procedure for bis-(4-oligoethyleneoxyphenyl)-4,4′-bipyridinium dichloride (EV1-EV3)

The synthesis of EV2 is described as an example (Scheme 1, Step 4); EV1 and EV3 were prepared in an identical manner. It was prepared by adding the 4-(2-(2-ethoxyethoxy) ethoxy] aniline (0.192 g, 0.85 mmol) to a round-bottomed flask containing Zincke salt (0.217 g, 0.39 mmol) and 15 mL of N,N-dimethylacetamide (DMAc). The reaction mixture was stirred at room temperature for 3 h. At the end of the reaction, the crude product was collected by simply gravity filtration and washed with acetone to give a pure product (0.162 g, 0.25 mmol) metathesis reaction. The salts EV1 and EV3 were synthesized from the metathesis reaction of the dichloride salts with lithium triflimide. The synthesis of 2 is described as an example (Scheme 1, Step 5); 1 and 3 were prepared in an identical manner. The lithium salt (0.316 g, 1.10 mmol) dissolved in 5 mL of deionized water was added to a reaction flask containing a clear solution of EV2 (0.284 g, 0.44 mmol) dissolved in 20 mL of ethanol. The flask was heated to reflux for 72 h. At the end of the reaction, the solvent ethanol was removed by using a rotary evaporator. The reaction mixture was then dissolved in chloroform and extracted from deionized water to give a pure brown product (0.466 g, 0.41 mmol).

### Example 2

Scheme 2 shows the chemical structures of additional extended viologen salts (OIPCs), which were prepared in an analogous manner to the 4-oligoethyleneoxy anilines of Example 1 using Zincke Salt and metathesis reactions.

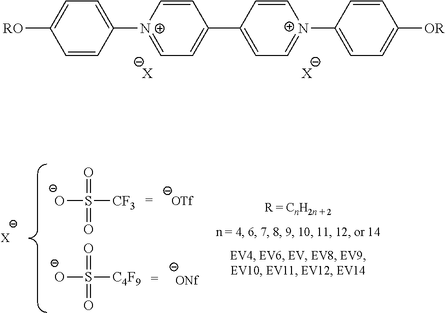

The synthetic schemes for the preparation of the bistriflates, and bisnonaflates of Scheme 2 are shown in Scheme 3, and Scheme 4, respectively. The procedures involved 5 steps: the preparation 1-bromoalkane starting from n-alkanol (Step 1), the preparation of n-alkoxyaniline (Step 2), the preparation of Zincke salt (Step 3), the preparation of extended viologen dichloride form from the reaction of Zincke salt with the n-alkoxyaniline (Step 4), exchange of dichloride extended viologen to bistriflates (Scheme 3), or bisnonaflates (Scheme 4) (Step 5).

Scheme 3 shows the synthesis of extended viologen bistriflate salts (EVₙSO₃CF₃), where EVₙ=C₄H₉ (EV4), C₆H₁₃ (EV6), C₇H₁₅ (EV7), C₈H₁₇ (EV8), C₉H₁₉ (EV9), C₁₀H₂₁ (EV10), C₁₁H₂₃ (EV11), C₁₂H₂₅ (EV12), and C₁₄H₂₉ (EV14).

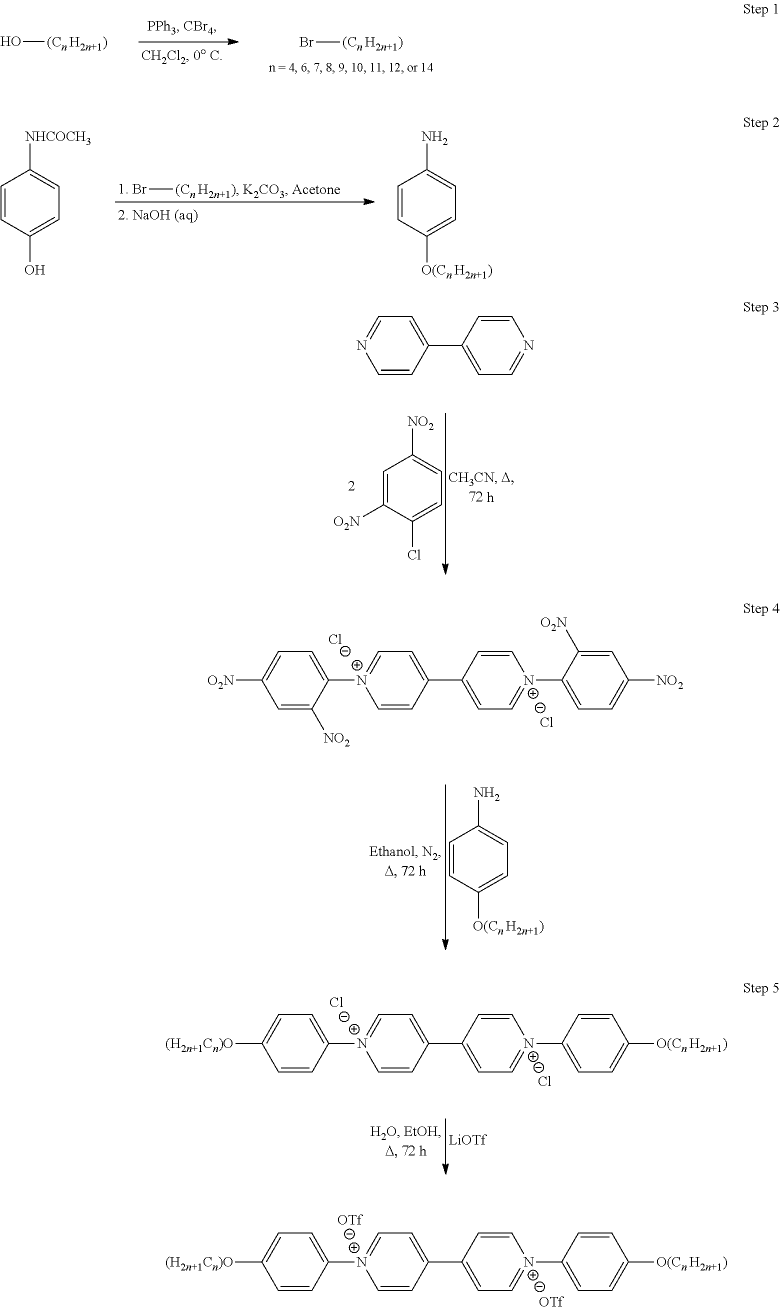

Scheme 4 shows the synthesis of extended viologen bisnonaflate salts (EVₙSO₃C₄F₉), where EVₙ=C₄H₉(EV4), C₆H₁₃ (EV6), C₇H₁₅ (EV7), C₈H₁₇ (EV8), C₉H₁₉ (EV9), C₁₀H₂₁ (EV10), C₁₁H₂₃ (EV11), C₁₂H₂₅ (EV12), and C₁₄H₂₉ (EV14).

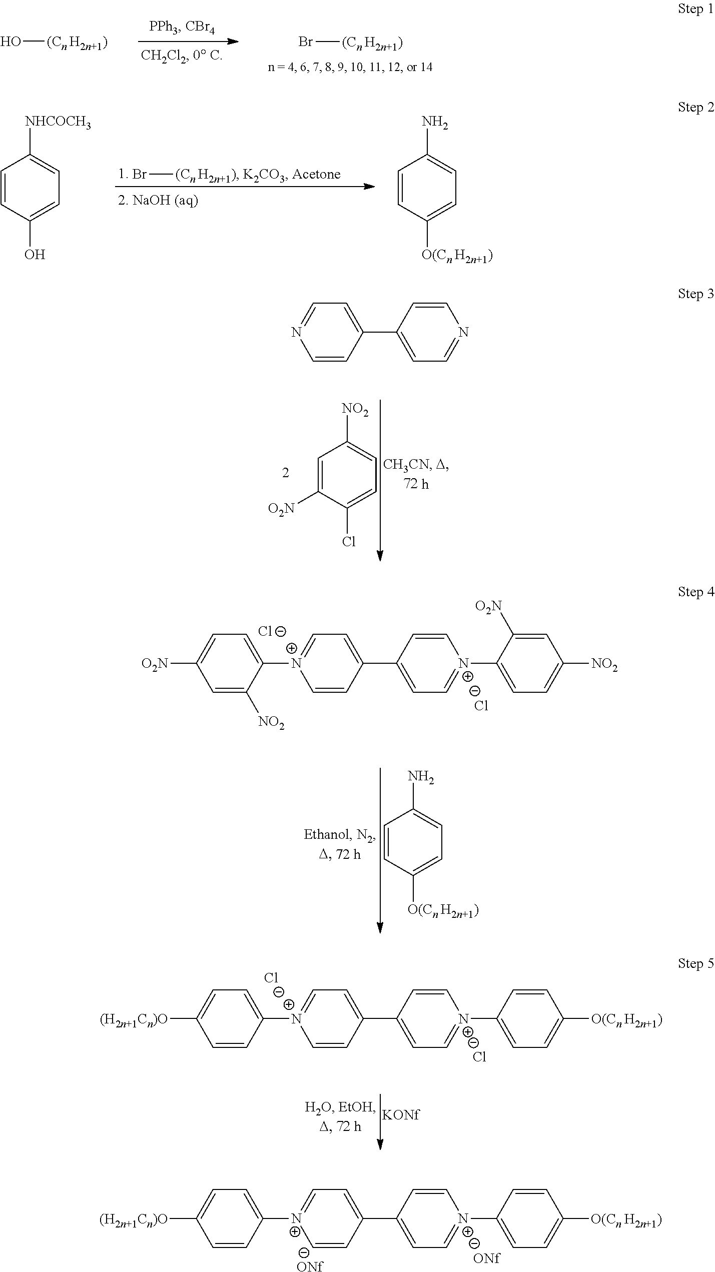

### Example 3

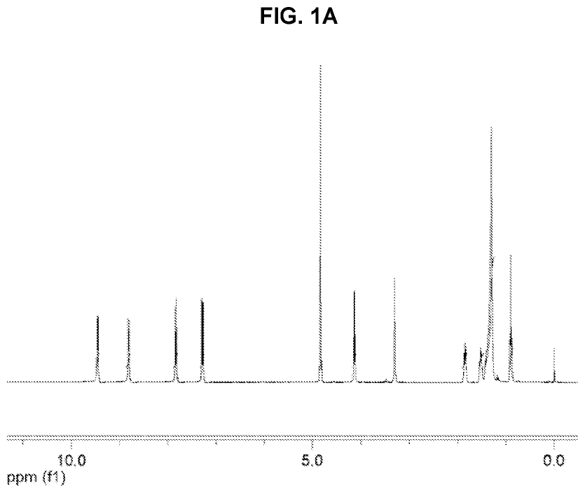

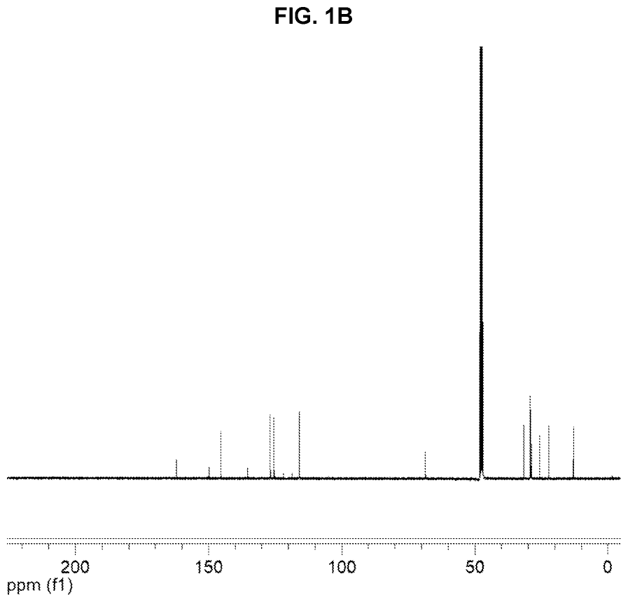

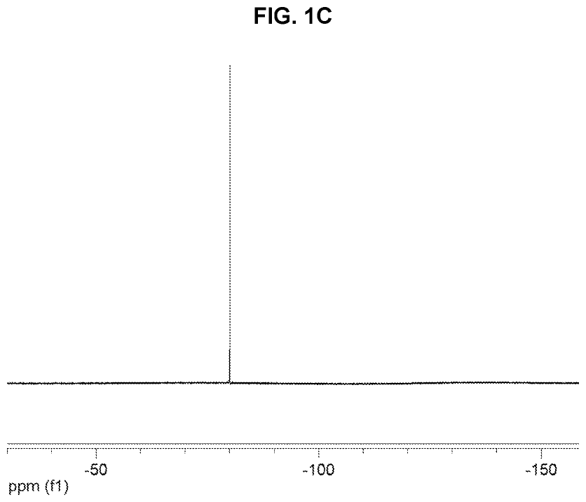

FIG. 1A-C show ¹H NMR (FIG. 1A), ¹³C NMR (FIG. 1B) and ¹⁹F NMR (FIG. 1C) spectra of EVₙSO₃CF₃, where EVₙ=C₁₂H₂₅ (EV12), in CD₃OD taken at room temperature. ¹H NMR spectrum data for EV₁₂SO₃CF₃: δ_{H} (CD₃OD, 400 MHz, ppm): 9.46 (4H, d, J=6.8 Hz), 8.82 (4H, d, J=6.8 Hz), 7.84 (4H, d, J=8.8 Hz), 7.29 (4H, d, J=9.2 Hz), 4.15 (4H, t, J=6.4 Hz), 1.88-1.81 (4H, m), 1.56-1.30 (36H, m), 0.92 (6H, t, J=6.4 Hz). ¹³C NMR spectrum data for EV₁₂SO₃CF₃: δ_{C} (CD₃OD, 100 MHz, ppm): 161.98, 149.80, 145.31, 135.28, 126.77, 126.41, 121.89, 118.72, 115.83, 68.53, 31.64, 29.34, 29.32, 29.28, 29.05, 29.03, 28.75, 25.66, 22.30, 13.00. ¹⁹F NMR spectrum data for EV₁₂SO₃CF₃: δ_{F} (CD₃OD, 376 MHz, ppm): −80.03.

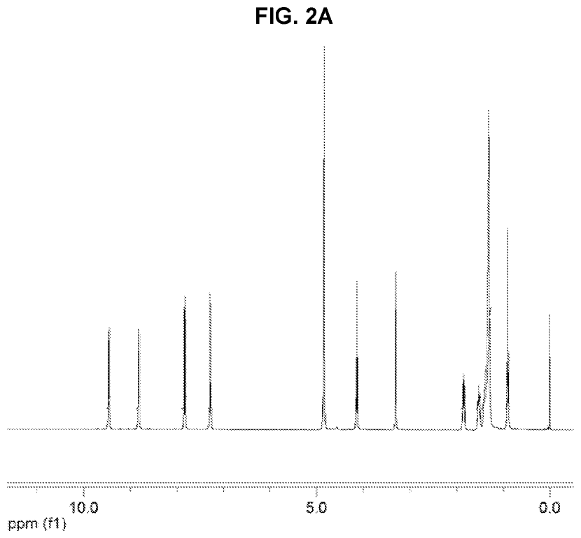

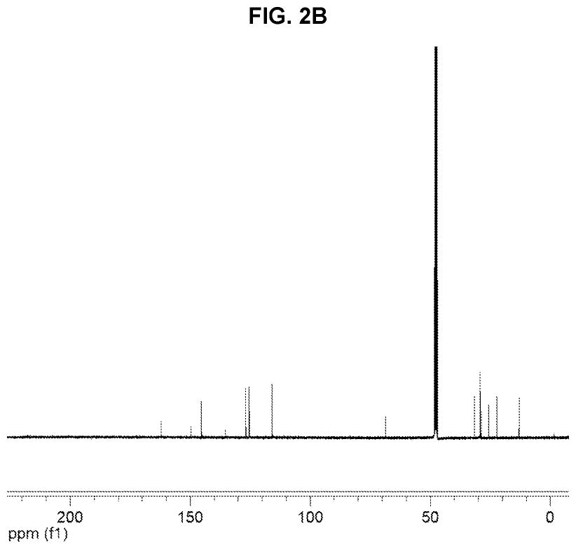

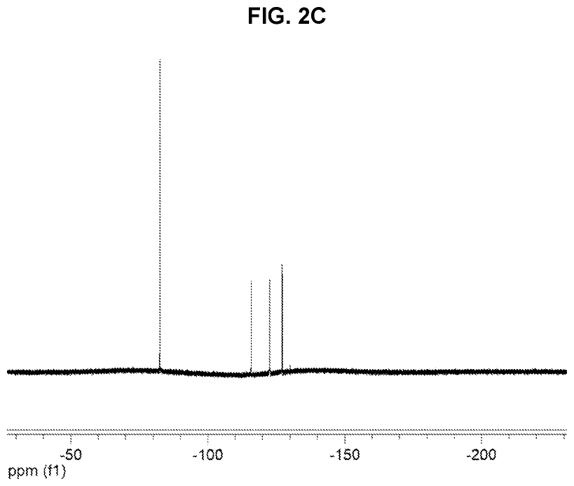

FIG. 2A-C show ¹H NMR (FIG. 2A), ¹³C NMR (FIG. 2B), and ¹⁹F NMR (FIG. 2C) spectra of EVₙSO₃C₄F₉, where EVₙ=C₁₂H₂₅ (EV12), in CD₃OD taken at room temperature. ¹H NMR spectrum data for EV₁₂SO₃C₄F₉: δ_{H} (CD₃OD, 400 MHz, ppm): 9.47 (4H, d, J=6.8 Hz), 8.82 (4H, d, J=6.8 Hz), 7.84 (4H, d, J=8.8 Hz), 7.29 (4H, d, J=9.2 Hz), 4.15 (4H, t, J=6.4 Hz), 1.89-1.82 (4H, m), 1.56-1.31 (36H, m), 0.92 (6H, t, J=6.4 Hz). ¹³C NMR spectrum data for EV₁₂SO₃C₄F₉: δ_{C} (CD₃OD, 100 MHz, ppm): 161.99, 149.78, 145.31, 135.26, 126.77, 125.39, 115.83, 68.52, 31.63, 29.34, 29.31, 29.28, 29.03, 28.74, 25.70, 22.29, 12.99. ¹⁹F NMR spectrum data for EV₁₂SO₃C₄F₉: δ_{F} (CD₃OD, 376 MHz, ppm): −82.52, −115.86, −122.67, −127.22.

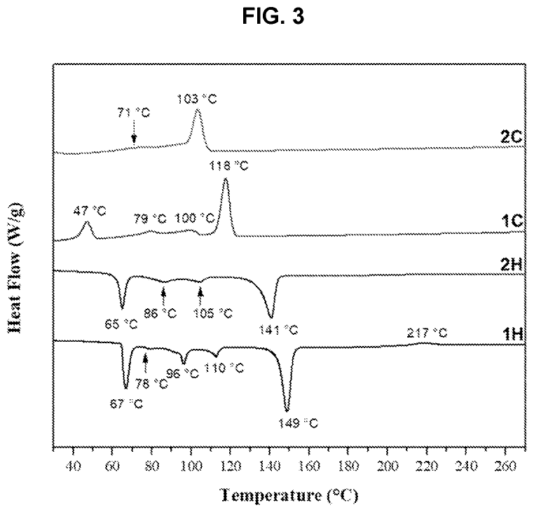

FIG. 3 shows differential scanning calorimetry (DSC) thermograms of EVₙSO₃CF₃, where EVₙ=C₁₀H₂₁ (EV10), obtained at heating and cooling rates of 10° C./min.

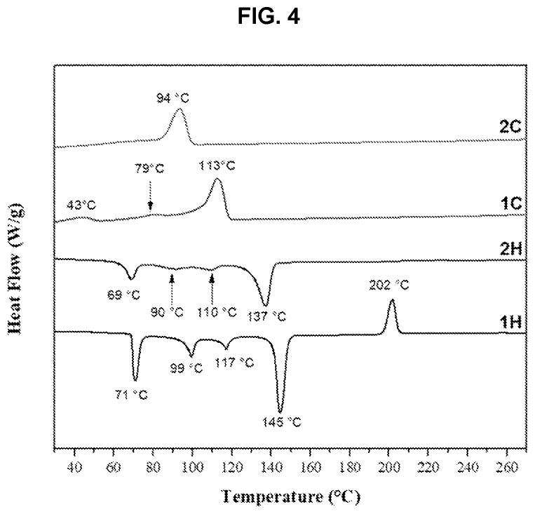

FIG. 4 shows DSC thermograms of EVₙSO₃CF₃, where EVₙ=C₁₂H₂₅ (EV12), obtained at heating and cooling rates of 10° C./min.

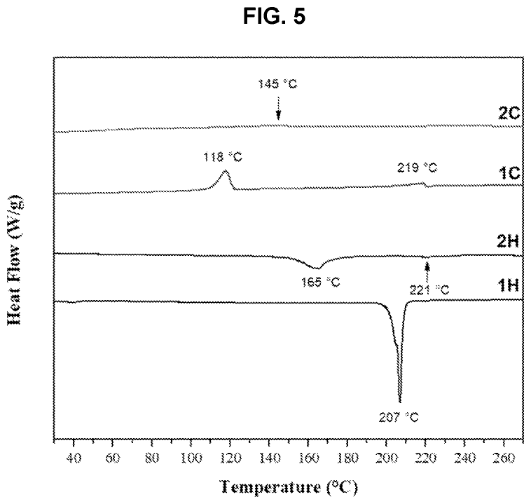

FIG. 5 shows DSC thermograms of EVₙSO₃C₄F₉, where EVₙ=C₁₀H₂₁ (EV10), obtained at heating and cooling rates of 10° C./min.

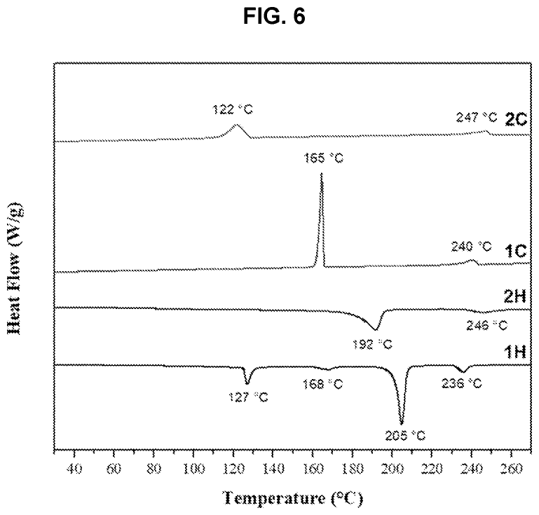

FIG. 6 shows DSC thermograms of EVₙSO₃C₄F₉, where EVₙ=C₁₂H₂₅ (EV12), obtained at heating and cooling rates of 10° C./min.

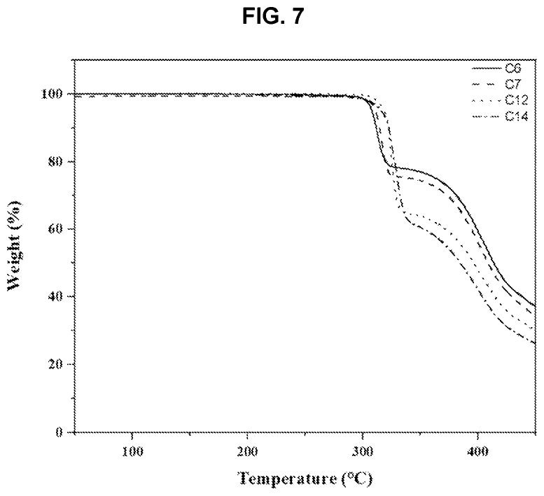

FIG. 7 shows thermogravimetric analysis (TGA) thermograms of EVₙSO₃CF₃, where EVₙ=C₆H₁₃ (EV6), C₇H₁₅ (EV7), C₁₂H₂₅ (EV12), and C₁₄H₂₉ (EV14), obtained at a heating rate of 10° C./min in nitrogen.

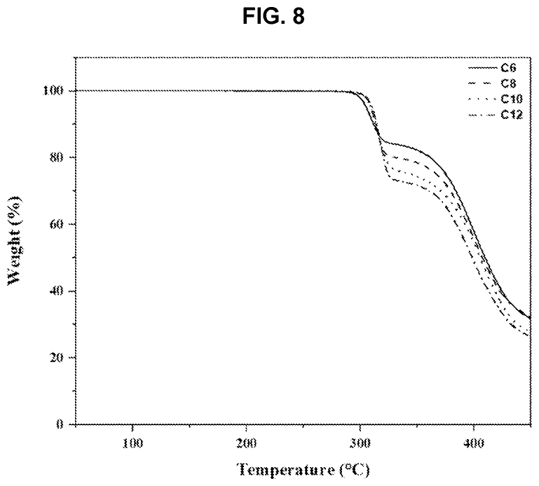

FIG. 8 shows TGA thermograms of EVₙSO₃C₄F₉, where EVₙ=C₆H₁₃ (EV6), C₈H₁₇ (EV8), C₁₀H₂₁ (EV10), and C₁₂H₂₅ (EV12), obtained at a heating rate of 10° C./min in nitrogen.

1. A solid-state electrolyte comprising:
a viologen salt of formula (I) 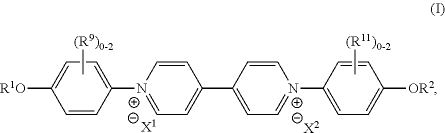
wherein:
^{Θ}X¹ and ^{Θ}X² are each independently 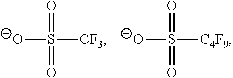
^{Θ}BF₄, ^{Θ}PF₆, ^{Θ}Br, ^{Θ}Cl, or 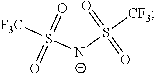 [SMILES: O=S(=O)([N-]S(=O)(=O)C(F)(F)F)C(F)(F)F]
R¹ and R² are each independently C₄₋₂₀alkyl or 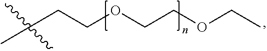
where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.

2. The solid-state electrolyte of claim 1, wherein R¹ is

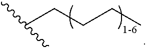

3. The solid-state electrolyte of claim 1, wherein R² is

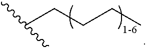

4. The solid-state electrolyte of claim 1, wherein the viologen salt is a symmetric viologen salt.

5. The solid-state electrolyte of claim 1, wherein ^{Θ}X¹ and ^{Θ}X² are each

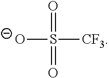

6. The solid-state electrolyte of claim 1, wherein ^{Θ}X¹ and ^{Θ}X² are each

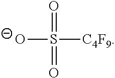

7. The solid-state electrolyte of claim 1, comprising a viologen salt of formula (I-a):

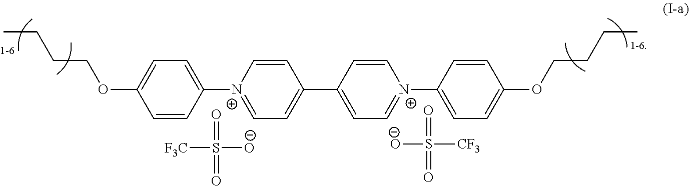

8. The solid-state electrolyte of claim 1, comprising a viologen salt of formula (I-b):

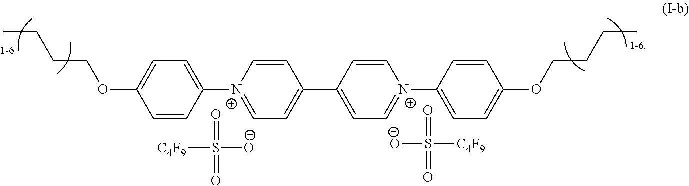

9. (canceled)

10. A solid-state battery comprising
a cathode;
an anode;
a separator; and
a solid-state electrolyte, wherein the solid-state electrolyte comprises:
a viologen salt of formula (I): 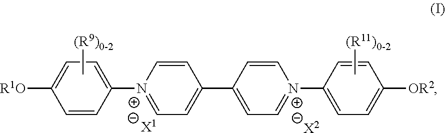
wherein:
^{Θ}X¹ and ^{Θ}X² are each independently 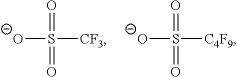
^{Θ}BF₄, ^{Θ}PF₆, ^{Θ}Br, ^{Θ}Cl, or 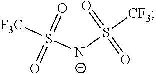 [SMILES: O=S(=O)([N-]S(=O)(=O)C(F)(F)F)C(F)(F)F]
R¹ and R² are each independently C₄₋₂₀alkyl or 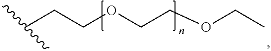
where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.

11. (canceled)

12. A solar cell comprising:
a n-type semiconductor layer;
a p-type semiconductor layer; and
a solid-state electrolyte, wherein the solid-state electrolyte comprises:
a viologen salt of formula (I): 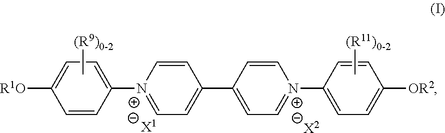
wherein:
^{Θ}X¹ and ^{Θ}X² are each independently 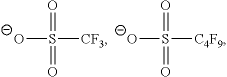
^{Θ}BF₄, ^{Θ}PF₆, ^{Θ}Br, ^{Θ}Cl, or 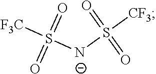 [SMILES: O=S(=O)([N-]S(=O)(=O)C(F)(F)F)C(F)(F)F]
R¹ and R² are each independently C₄₋₂₀alkyl or 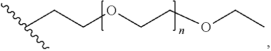
where n is 0 to 2;
R⁹, at each occurrence, is independently C₁₋₄alkyl, C₁₋₂haloalkyl, —OC₁₋₄alkyl, —OC₁₋₂haloalkyl, halogen, —NO₂, or —CN; and
R¹¹, at each occurrence, is independently C₁₋₄alkyl, C₁₋₂haloalkyl, —OC₁₋₄alkyl, —OC₁₋₂haloalkyl, halogen, —NO₂, or —CN.

13. (canceled)

14. The solid-state battery of claim 10, wherein R¹ is

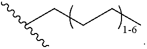

15. The solid-state battery of claim 10, wherein R² is

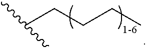

16. The solid-state battery of claim 10, wherein the viologen salt is a symmetric viologen salt.

17. The solid-state battery of claim 10, wherein ^{Θ}X¹ and ^{Θ}X² are each

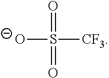

18. The solid-state battery of claim 10, wherein ^{Θ}X¹ and ^{Θ}X² are each

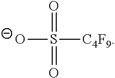

19. The solid-state battery of claim 10, wherein the solid-state electrolyte comprises a viologen salt of formula (I-a):

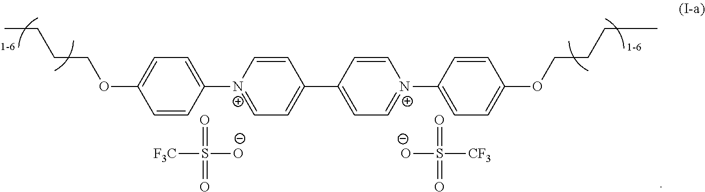

20. The solid-state battery of claim 10, wherein the solid-state electrolyte comprises a viologen salt of formula (I-b):

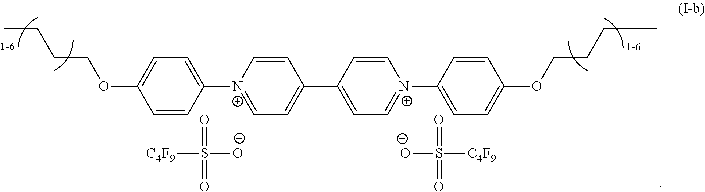

21. The solar cell of claim 12, wherein R¹ is 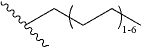
and R² is

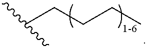

22. The solar cell of claim 12, wherein the viologen salt is a symmetric viologen salt.

23. The solar cell of claim 12, wherein ^{Θ}X¹ and ^{Θ}X² are each [chemical structure]
